(19) United States
(12) Patent Application Publication
(10) Pub. No.: US 2022/0312776 A1
(43) Pub. Date: Oct. 6, 2022

(54) METHODS OF INCREASING THE GERMINATION RATE OF FUNGAL SPORES

(71) Applicant: Bayer CropScience Biologics GmbH, Wismar (DE)

(72) Inventors: Daniel LANVER, Lübow (DE); Sabine EGGERS, Kaltenhof/Poel (DE); Stefanie ADAM, Brandenhusen/Poel (DE)

(21) Appl. No.: 17/616,001

(22) PCT Filed: Jun. 3, 2020
(86) PCT No.: PCT/EP2020/065288

§ 371 Date: Dec. 2, 2021

(30) Foreign Application Priority Data
Jun. 7, 2019 (EP) 19179012.0

(51) Int. Cl. A01N 63/30; C12N 1/14; A01N 25/02; A01N 25/22

(52) U.S. Cl. CPC A01N 63/30; C12N 1/145; A01N 25/02; A01N 25/22

(57) ABSTRACT

The present invention relates to a method of increasing the germination rate of spores of a fungal microorganisms formulated in a liquid composition essentially free of water, comprising, prior to applying to a plant, plant part or locus where a plant is growing or intended to be grown, suspending said composition in an agriculturally acceptable water-based liquid in a ratio of liquid composition and water-based liquid of between 1000:1 and 1:10. The invention further relates to a method of increasing efficacy of a biological control agent based on spores of a fungal microorganisms and a method of controlling the quality of such biological control agents.

Biological control agents become more and more important in the area of plant protection, be it for combatting various fungal or insect pests or for improving plant growth. Although also viruses are available which can be used as biological control agents, mainly those based on bacteria and fungi are used in this area so far. The most prominent form of biological control agents based on fungi are their asexual spores called conidia as well as blastospores, but also other fungal propagules may be promising agents, such as (micro)sclerotia, ascospores, basidiospores, chlamydospores or hyphal fragments.

Fungal spores such as those to be applied in biological plant protection are usually formulated in a dried state in order to inactivate their metabolism and thus to achieve a reasonable shelf stability. The spores are usually reactivated by suspending them in water prior to application. This step may cause imbibition damage which may lead to the death of conidia. Imbibition damage is not a novel phenomenon but was studied in many desiccated organisms e.g. pollen, seeds and yeasts (see e.g. Crowe et al., 1992. Anhydrobiosis. Annual Review of Physiology 1992 54:1, 579-599) as well as in fungal conidia (see e.g. Faria et al., 2017. Susceptibility of the biocontrol fungi Metarhizium anisopliae and Trichoderma asperellum (Ascomycota: Hypocreales) to imbibitional damage is driven by conidial vigor. Biological Control 107 (2017) 87-94). Unlike many spores based on bacteria, such as Bacillus spores, many fungal spores are less robust and it has proven to be difficult to provide fungal spores in a form which meets the needs of commercial products. Another issue is the provision of fungal spore formulations with constantly high reactivation rates of spores. Accordingly, fungal spores are normally not available as unformulated spore powder but in formulated form where the formulation is adapted to provide improved stability and shelf-life of the formulation, i.e. high germination rates of the fungal spores. Despite the efforts in optimizing stability of fungal spores including germination rate, there is still the need to provide for a general method of improving the germination rate of formulated fungal spores in a species-independent manner.

This technical problem has at least partially been solved by the present invention.

Accordingly, in one aspect, the present invention relates to a method of increasing the germination rate of spores of a fungal microorganisms formulated in a liquid composition essentially free of water, comprising, prior to applying to a plant, plant part or locus where a plant is growing or intended to be grown, suspending said composition in an agriculturally acceptable water-based liquid in a ratio of liquid composition:water-based liquid of between 1000:1 and 1:10.

In the context of the present invention, the “germination rate” denotes the ability of fungal spores to germinate. % germination rate accordingly means the percentage of fungal spores which is able to germinate under given conditions. Methods of measuring the germination rate are well-known in the art.

For example, spores are spread onto the surface of an agar medium, and the proportion of spores developing germ tubes is determined microscopically after a time which is dependent on the growth of that species which usually varies between about 6 and about 48 hours incubation at appropriate growth temperatures (Oliveira et al., 2015. A protocol for determination of conidial viability of the fungal entomopathogens Beauveria bassiana and Metarhizium anisopliae from commercial products. Journal of Microbiological Methods 119; pp: 44-52, and references therein). However, the most preferred method is that according to the present invention which is also the method of increasing the germination rate as compared to other methods. The term “germination rate” is not to be confused with the term “viability” which relates to the maximum germination rate of fungal spores which could be achieved using the ideal method of reactivation. That is to say that the method according to the present invention is closer to detecting viability of fungal spores than any other method published so far for fungal spores formulated as described herein.

Due to the sensitivity of dormant structures such as fungal spores, formulations comprising a low concentration of water or even being essentially free of water are a preferred formulation type for fungal spores in agronomy. Accordingly, said liquid composition comprising fungal spores is essentially free of water. If water is present in such formulations, it mainly comes from water in the dried spore powder or traces of water in the other formulants. The higher the amount of spore powder the higher the water content may be. Water concentrations of between 0.1 wt.-% and 12 wt.-%, such as between 0.3 wt.-% and 8 wt.-%, or between 4 wt.-% and 7 wt.-% are possible due to these facts, which range would then fall within the definition of “essentially free of water”. The amount of spore powder in the liquid composition also depends on the application so that a composition for use in nematode control may need a higher spore concentration than one for use for increasing plant growth in general. Accordingly, exemplary water concentrations include up to 1 wt.-%, up to 2 wt.-%, up to 3 wt.-%, up to 4 wt.-%, up to 5 wt.-%, up to 6 wt.-%, up to 7 wt.-%, up to 8 wt.-%, up to 9 wt.-%, up to 10 wt.-%, up to 11 wt.-% and up to 12% of the liquid composition comprising fungal spores which all fall within the definition of “essentially free of water”. In other words, “essentially free of water” means a water content in the liquid composition of 12% or less, preferably 10% or less, even more preferably 8% or less such as 7% or less or 6% or less. This water content of 12 wt.-% or less of the composition is also denominated “residual water”. As indicated above, such residual water is comprised in the ingredients of the composition of the invention which means that it is not added as a separate ingredient. Accordingly, the residual water content of the liquid composition is 8 wt.-% or less, such as any of the above values. Phrased in a different way with regard to the residual water content in the spores, said residual water content in the fungal spores may be 30 wt.-% or less, preferably 20 wt.-% or less, more preferably 15 wt.-% or less, such as 12 wt.-% or less, for example 10 wt.-% or less, 8 wt.-% or less, 6 wt.-% or less or even 4 wt.-% or less. With this definition, the water content of the liquid composition not taking into account the fungal spores is less than 1 wt.-%. Also this alternative definition falls within the term “essentially free of water”. For example, in a liquid composition containing 20 wt.-% fungal spores which is otherwise free of water and where the spores have a residual water content of 10%, the resulting water content is 2%.

For the sake of clarity, whereas the added percentages of fungal spores and other ingredients, such as at least one surfactant as described further below, shall not exceed 100%, the residual water content may be given in the liquid composition without adding up to the former ingredients due to said “residual water” being comprised in the other ingredients.

The water content of the spore powder prior to addition into the formulation according to the invention may be measured according to methods well-known in the art, e.g. using a moisture meter such as one available from Sartorius (Type MA 30).

As far as not otherwise defined, % in the present application refers to wt.-%.

An agronomically acceptable water-based liquid relates to a liquid which the farmer or any user of an agricultural product uses in order to dilute a formulation comprising an agent based on microorganims to be applied onto plants, in the present case a liquid composition as defined elsewhere to the desired concentration prior to applying it to the field. In most cases, such water-based liquid is water. However, in certain cases, also additives like fertilizers or other substances or formulations may be added to the water or the water may contain trace amounts of organic matter, minerals, colloids or any other chemicals.

The invention also relates to a method of increasing efficacy of spores of a fungal microorganisms and formulated in a liquid composition essentially free of water, in agriculture, comprising, prior to applying to a plant, plant part or locus where a plant is growing or intended to be grown, suspending said composition in an agriculturally acceptable water-based liquid in a ratio of liquid composition:water-based liquid of between 1000:1 and 1:10.

Efficacy as used in the present application is the beneficial action of a biological control agent on a plant or the locus where the plant is growing or intended to be grown. For example, efficacy can be nematicidal, fungicidal or pesticidal action of a biological control agent. Increased efficacy of a biological control agent based on fungal spores is, inter alia, highly dependent on the germination rate and/or viability of spores because the higher said viability or germination rate the more spores will be able to germinate and continue growth in order to exert its potential, e.g. to infect its host organisms and/or control other microorganisms and/or colonize a plant to improve plant growth and/or plant health as described elsewhere. Efficacy is preferably increased as compared to a plant or locus where the plant is growing or intended to be grown which has not been treated with said biological control agent. More preferably, efficacy is increased as compared to a plant or locus where the plant is growing or intended to be grown which has been treated with a suspension of a liquid composition comprising a biological control agent based on fungal spores formulated in a liquid composition essentially free of water in a ratio of liquid composition:water-based liquid of between 1000:1 and 1:10. Rather, a preferred control method comprises suspension of said liquid composition in a water-based liquid such as water in a ratio of 1:99.

In a further aspect, the present invention relates to a method of controlling the quality of spores of a fungal microorganism formulated in a liquid composition essentially free of water, comprising suspending a sample of said composition in a water-based liquid in a ratio of liquid composition:water-based liquid of between 1000:1 and 1:10 and cultivating a part of the resulting suspension. Quality is normally controlled after production, i.e. in this case after fermentation, processing and mixing of fungal spores into a liquid composition as defined herein, but also after defined storage time in order to ensure sufficient viability of said spores.

The term “quality” as used in connection with the present invention relates to the achievable germination rate of fungal spores produced and mixed into a liquid composition as described herein after production and prior to application. Said quality is tested under optimal conditions, i.e. preferably by using the method of the present invention.

In the course of the present invention, it has surprisingly been found that fungal spores, when present in a liquid composition essentially free of water, can be reactivated more efficiently when treated according to the methods of the present invention. In particular, it was found that resuspension in a water-based liquid in a ratio of liquid composition:water-based liquid of between 1000:1 and 1:10, preferably in a ratio of between 100:1 and 1:10, leads to an increased germination rate of fungal spores as compared to fungal spores in a liquid composition resuspended in a ratio greater than 1:10, such as 1:99. That means that a greater fraction of the dormant spores are able to germinate after resuspension using the present method as compared to spores in suspension created with a smaller ratio of liquid composition and water-based liquid. This finding enables for a more reliable quality control for production batches of formulations comprising fungal spores because the increased germination rate is closer to overall viability. Apart from the production process which has a great influence on the quality/viability of fungal spores in general and without wishing to be bound by any scientific theory in this regard, the inventors consider that liquid composition essentially free of water, more particularly the presence of surfactant as described further below, and even more particularly comparably high concentrations of surfactant, can interfere with the germination rate of fungal spores and their capacity to be revived. The present invention also facilitates application of liquid compositions comprising fungal spores in agriculture. In conncention with the present application, suspending may be effected using any known means, such as by shaking or stirring.

In a preferred embodiment, said liquid composition comprises at least one surfactant.

The term “at least one” indicates that in any case one surfactant is present in the liquid composition. However, more than one such as (at least) two, (at least) three, (at least) four, (at least) 5 or even more surfactants may be present in the liquid composition.

Surfactants are often used in agricultural formulations to ensure proper suspension or emulsion of the formulation in water prior to application in the field or the greenhouse. However, only low concentrations of surfactants are normally contained in liquid compositions as they are sufficient to provide the desired effect. Recently, novel liquid compositions for fungal spores have been developed comprising higher concentrations of surfactants, as described further below.

Said at least one surfactant may be any surfactant which can be used in agriculture and which is compatible with fungal spores. Testing of compatibility is well within the knowledge of the person skilled in the art and may be effected by mixing fungal spores with a surfactant as described further below and testing the resulting germination rate as compared to a mixture not containing said surfactant.

Non-ionic and/or anionic surfactants are all substances of this type which can customarily be employed in agrochemical agents. Possible nonionic surfactants are selected from the groups of polyethylene oxide-polypropylene oxide block copolymers, ethoxylated mono-, di- and/or triglycerides where ethoxylated castor oil or ethoxylated vegetable oils may be mentioned by way of example, polyethylene glycol ethers of branched or linear alcohols, reaction products of fatty acids or fatty acid alcohols with ethylene oxide and/or propylene oxide, furthermore branched or linear alkylaryl ethoxylates, where polyethylene oxide-sorbitan fatty acid esters may be mentioned by way of example. Out of the examples mentioned above selected classes can be optionally phosphated and neutralized with bases. Possible anionic surfactants are all substances of this type which can customarily be employed in agrochemical agents. Alkali metal, alkaline earth metal and ammonium salts of alkylsulphonic or alkylphosphoric acids as well as alkylarylsulphonic or alkylarylphosphoric acids are preferred. A further preferred group of anionic surfactants or dispersing aids are alkali metal, alkaline earth metal and ammonium salts of polystyrenesulphonic acids, salts of polyvinylsulphonic acids, salts of alkylnaphthalene sulphonic acids, salts of naphthalenesulphonic acid-formaldehyde condensation products, salts of condensation products of naphthalenesulphonic acid, phenolsulphonic acid and formaldehyde, and salts of lignosulphonic acid. A further preferred group of anionic surfactants or dispersing aids are alkali metal, alkaline earth metal and ammonium salts of sarcosinates or taurates.

The liquid composition comprising fungal spores may further comprise at least one antifoaming agent in order to prevent foaming upon dilution with water, in particular where said liquid composition comprises a substance which acts as surfactant. Suitable antifoaming agents are e.g. paraffinic oils, vegetable oils, silicone oils (e.g. Silcolapse 411, Silcolapse 454, Silcolapse 482 from Solvay; Silfoam SC1132, Silfoam SC132 from Wacker; Xiameter ACP-0100 from Dow) or aqueous silicone oil emulsions (e.g. SAG30, SAG 1572/Momentive, Silcolapse 426R, Silcolapse 432/Solvay; Silfar SE4/Wacker; Antifoam 8830/Harcros Chemicals). In a preferred embodiment the concentration of antifoaming agents is in the range of 0 to 0.5% wt, e. g. of 0.1 to 0.3% wt. In particular, the concentration of antifoaming agent may be 0, 0.1, 0.2, 0.3, 0.4 or 0.5% wt or any value in between.

Fungal spores as within the scope of the present invention comprise asexual spores, such as conidia as well as blastospores, but also other fungal propagules such as ascospores, basidiospores, chlamydospores. (micro)sclerotia, although not being spores in the strict sense, may also be used within the scope of the invention. Preferably, the spores are conidia. Conidia are a kind of spores asexually formed by many fungal microorganism useful in agriculture, e.g. of the genus Purpureocillium, Isaria, Metarhizium, Beauveria, Trichoderma and Penicillium.

The amount or concentration of spores in the liquid composition is not particularly limited. However, in order to achieve the desired effect, the liquid composition should comprise at least 1 wt.-% of fungal spores, preferably at least 5 wt.-%. Depending on the fungal species used and the other components of the liquid composition, the amount/concentration of fungal spores may be up to about 30 wt.-%.

The temperature of the liquid composition and/or the water-based liquid may have a further beneficial effect on the germination rate of fungal spores. In a preferred embodiment, the temperature of the water-based liquid in which to re-suspend the liquid composition comprising fungal spores which is essentially free of water is at least 12° C., preferably at least 20° C. such as at least 22° C.

In another preferred embodiment, liquid compositions, in particular those stored under cold conditions, are adjusted prior to suspending it in said water-based liquid to between about 12° C. and about 41° C., preferably at least 20° C. such as at least 22° C., such as 15° C., 20° C., 21° C., 22° C., 23° C., 24° C., 25° C., 26° C., 27° C., 28° C., 29° C., 30° C., 31° C., 32° C., 33° C., 34° C., 35° C., 36° C., 37° C. and 38° C. This will improve the germination rate, more particularly if the ratio of liquid composition:water-based liquid is high, such as at least 1:1.

Alternatively or in addition, the temperature of the water-based liquid may be chosen at any value in between 12° C. and 41° C., preferably 40° C., more preferably 39° C., such as 15° C., 20° C., 21° C., 22° C., 23° C., 24° C., 25° C., 26° C., 27° C., 28° C., 29° C., 30° C., 31° C., 32° C., 33° C., 34° C., 35° C., 36° C., 37° C. and 38° C. and any temperature in between. A preferred range includes temperatures between about 20° C. and 40° C., more preferably between about 20° and about 37° C. or about 22° C. and about 37° C.

Such adjusted temperature for the liquid composition and/or the water-based liquid may have a further beneficial effect on the germination rate of fungal spores. The temperature used will depend on the fungal species and may be chosen accordingly. It is believed that a temperature of 42° C. and above will not result in a further increase of viability and/or efficacy for fungal spores of most species used in agronomy as proteins start to denaturate at this temperature.

Preferably, said water-based liquid has a temperature of at least 25° C. It is more preferred that in this embodiment, the fungal microorganism is Purpureocillium lilacinum. It is even more preferred that the water-based liquid has a temperature of at least 35° C., such as 36° C., 37° C. or 38 ° C., most preferably also in connection with said Purpureocillium lilacinum strain. In connection with all preferred embodiments in this aspect, the Purpureocillium lilacinum strain is most preferably strain 251.

In one preferred embodiment, said liquid composition has essentially the same temperature as said water-based liquid. In this context, the term “essentially” denotes a temperature of the liquid composition that does not differ by more than 5° C. from that of the water-based liquid.

For liquid compositions stored under cold conditions, it may be advantageous, prior to addition of the water-based liquid, to adjust the temperature of said liquid composition to or close to that of the water-based liquid.

Liquid compositions comprising fungal spores are well-known in the art. Whereas most formulations comprise only low amounts of surfactant as described herein, such as between 0.001 and 0.5 wt.-%, there are also novel formulations available where the amount of such surfactant is much higher, see e.g. WO2012/163322 or WO2016/050726, both incorporated herein by reference in their entirety, as it turned out that such formulations provide a longer storage stability of fungal spores as compared to previously known formulations. The present inventors have found that in particular in compositions comprising high amounts of such surfactants the germination rate of fungal spores can be further increased.

In a preferred embodiment the concentration of said at least one surfactant as defined herein ranges between 1 and 98 wt.-%, such as between 10 and 96 wt.-%, 20 and 96 wt.-% or 40 and 96 wt.-%. More preferably, the amount of said at least one surfactant is at least 50% or ranges between 50 and 96 wt.-%, such as between 70 and wt.-90% or between 65 and 85 wt.-%. Accordingly, the composition according to the present invention may comprise 50, 55, 60, 65, 70, 75, 80, 85, 90 or wt.-95% of said at least one surfactant and any value in between such as 71, 72, 73, 74, 76, 77, 78, 79, 81, 82, 83, 84, 86, 87, 88 and 89 wt.-%.

It is further preferred that in a liquid composition comprising spores of Purpureocillium lilacinum, in particular P. lilacinum strain 251, as biological control agent, the concentration of surfactant, which is more preferably a polyether-modified trisiloxane as disclosed in WO2012/163322 and most preferably a mixture of polyether-modified trisiloxane and fumed or precipitated silica as disclosed in WO2016/050726 (both of which are incorporated herein in their entirety) ranges between 60 and 85 wt.-%, preferably between 65 and 75 wt.-%, such as 66 wt.-%, 67 wt.-%, 68 wt.-%, 69 wt.-%, 70 wt.-%, 71 wt.-%, 72 wt.-%, 73 wt.-% or 74 wt.-% or any value in between.

In a preferred embodiment of the method of quality control according to the present invention said water further comprises an agent which decreases or prevents hyphal growth. This ingredient serves to facilitate counting spores and distinguishing viable from non-viable spores, where hyphal growth would cover the spores. In a more preferred embodiment, said agent belongs to the chemical class of benzimidazole carbamates having fungistatic activities. Most preferably, said agent is benomyl.

In a preferred embodiment, said suspension is to be held for at least 5 seconds prior to application or cultivation. The term “to be held” includes both the suspension to sit and to be agitated, depending on the circumstances the determination of which is well within the capabilities of the skilled person. Depending on the fungal species, the suspension may be held up to 36 hours prior to application or cultivation. Preferably, said suspension is to be held for at least 10 s, more preferably at least 30 s, even more preferably at least 1 min. Preferably, said suspension is held up to 12 hours, more preferably up to 6 hours. This includes times such as 2 min, 3 min, 4 min, 5 min, 10 min 15 min, 20 min, 25 min, 30 min, 60 min, 90 min or any value in between, including all real numbers. This should provide sufficient time for the dried fungal spores to initiate rehydration. During the holding time, the suspension may be left as is which will result in a temperature change towards the surrounding temperature. Alternatively and depending on the fungal species the suspension may be kept at the initial temperature resulting from suspending the liquid composition comprising fungal spores in the water-based liquid of a specific temperature as defined above.

In the course of the present invention it was surprisingly found that the ratio of composition and water-based liquid is important for the ability of the fungal spores to germinate and should be at least 1:10 and preferably higher, such as between 100:1 and 1:5, such as 50:1, 10:1, 5:1, 2:1, 1:1, 1:2 or 1:3 and any ratio in between. In a more preferred embodiment, the ratio of liquid composition and water-based liquid is between 20:1 and 1:5, such as between 10:1 and 1:2, such as 9:1, 8:1, 7:1, 6:1, 5:1, 4:1, 3:1, 2:1 or 1:1.

In a preferred embodiment, the methods of the invention further comprise, after said suspending, further diluting the suspension. Such dilution may be effected to come to the final concentration of the fungal active ingredient to be applied in the field or the greenhouse which may well be in a ratio of between 1:10 and 1:10000 (ratio of original liquid composition and water-based liquid). The person skilled in the art is well able to calculate the extent of the remaining dilution step. For example, with an initial suspension in a ratio of 1:1 with a desired final dilution of 1:99, the second dilution step needs to take place in a ratio of suspension obtained after first dilution and water-based liquid of 1:50. For the sake of completeness, the water-based liquids used in the first and the second dilution may differ in their composition.

In a more preferred embodiment, said at least one surfactant is a polyether-modified trisiloxane. Such polyether-modified trisiloxane preferably has the formula I

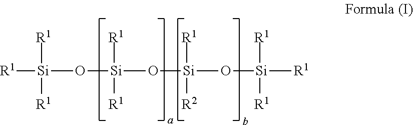

where

R¹ represents independent from each other identical or different hydrocarbyl radicals having 1-8 carbon atoms, preferred methyl-, ethyl-, propyl- and phenyl radicals, particularly preferred are methyl radicals.

a=0 to 1, preferred 0 to 0.5, particularly preferred 0,

b=0.8 to 2, preferred 1 to 1.2, particularly preferred 1,

in which: a+b<4 and b>a, preferred a+b<3 and particularly preferred a+b<2.

R² represents independent from each other identical or different polyether radicals of general formula (II)

R³O[CH₂CH₂O]_{c}[CH₂CH(CH₃)O]_{d}[CHR⁴CHR⁴O]ₑR⁵ Formula (II)

R³=independent from each other identical or different, bivalent hydrocarbyl radicals having 2-8 carbon atoms, which are optionally interrupted by oxygen atoms, preferred rest is the general formula (III) when n=2-8, particularly preferred —CH₂—CH₂—CH₂—,

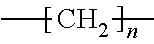

R⁴=independent from each other identical or different hydrocarbyl radicals having 1-12 carbon atoms or hydrogen radical, preferably a methyl-, ethyl-, phenyl- or a hydrogen radical.

R⁵=independent from each other identical or different hydrocarbyl radicals having 1-16 carbon atoms, which are optionally contain urethane functions, carbonyl functions or carboxylic acid ester functions, or hydrogen radical, preferred methyl or H, particularly preferred H.

C=0 to 40, preferred 1 to 15, particularly preferred 2 to10

d=0 to 40, preferred 0 to 10, particularly preferred 1 to 5

e=0 to 10, preferred 0 to 5, particularly preferred 0,

in which c+d+e>3

The polyether-modified trisiloxanes described above can be prepared by methods well known to the practioner by hydrosilylation reaction of a Si—H containing siloxane and unsaturated polyoxyalkylene derivatives, such as an allyl derivative, in the presence of a platinum catalyst. The reaction and the catalysts employed have been described for example, by W. Noll in “Chemie and Technologie der Silicone”, 2^{nd} ed., Verlag Chemie, Weinheim (1968), by B. Marciniec in “Appl. Homogeneous Catal. Organomet. Compd. 1996, 1, 487). It is common knowledge that the hydrosilylation products of SiH-containing siloxanes with unsaturated polyoxyalkylene derivatives can contain excess unsaturated polyoxyalkylene derivative.

Examples of water soluble or self-emulsifyable polyether-modified (PE/PP or block-CoPo PEPP) trisiloxanes include but are not limited to those described by CAS-No 27306-78-1 (e.g. Silwet L77 from MOMENTIVE), CAS-No 134180-76-0 (e.g. BreakThru S233 or BreakThru S240 from Evonik), CAS-No 67674-67-3 (e.g Silwet 408 from WACKER), other BreakThru-types, and other Silwet-types.

Preferred polyether-modified trisiloxanes include those described by CAS-No 134180-76-0, in particular Break-Thru S240 or Break-Thru S245, the latter of which being composed of Break-Thru S240 and fumed silica (Aerosil). In one preferred embodiment, the polyether-modified trisiloxane has the chemical denomination oxirane, mono(3-(1,3,3,3-tetramethyl-1-((trimethylsilyl)oxy)disiloxanyl)propyl)ether.

Fumed silica or precipitated silica as described in detail in WO2016/050726 may be comprised in the liquid composition in order to prevent (irreversible) sedimentation. Such agent builds a network within the polyether-modified trisiloxane which prevents or at least severely reduces spore sedimentation and does not influence viability of the spores.

The silica concentration in the liquid composition may range between 0.1 to 9 wt.-%, e. g. of 3 to 7 or 4 to 6 wt.-%. In one preferred embodiment, e.g. where spores of Purpureocillium lilacinum are used, the silica concentration is at least 5wt.-%. Alternatively it may range between 5 and 7 wt.-%. In particular, the silica concentration may be at least 0.1 wt.-%, at least 0.2 wt.-%, at least 0.5 wt.-%, at least 1 wt.-%, at least 1.5 wt.-%, at least 2 wt.-%, at least 2.5 wt.-%, at least 3 wt.-%, at least 4 wt.-%, at least 4.5 wt.-% at least 5 wt.-%, at least 5.5 wt.-%, at least 6 wt.-%, at least 6.5 wt.-%, at least 7 wt.-%, at least 7.5 wt.-%, at least 8 wt.-%, at least 8.5 wt.-% or at least 9 wt.-% as well as any specific of the foregoing values and essentially depends on the physical properties of the biological control agent as well as those of the carrier. In general, the silica concentration in the liquid composition may also depend on the fungal species, e.g. on the size of the fungal spores. Bigger spores are believed to necessitate less silica in order to prevent sedimentation.

In another preferred embodiment, the surfactant is Tween 20 (Polyoxyethylene(20)-sorbitan-monolaurate). Also here, fumed or precipitated silica such as Aerosil 200, may be added.

In another preferred embodiment, the composition comprises a petroleum-based liquid, such as e.g. in the product Met52 EC by Novozymes.

Any fungal species may be applied for the present invention. It is, however, preferred that said fungal spores are from a fungal species which is useful in agriculture, that means effective as biological control agent in plant protection or a plant growth promoting agent. More preferably, said fungus is a filamentous fungus.

The term “plant growth” generally comprises various sorts of improvements of plants that are not connected to the control of pests or phytopathogens. For example, advantageous properties that may be mentioned are improved crop characteristics including: emergence, crop yield, protein content, oil content, starch content, more developed root system, improved root growth, improved root size maintenance, improved root effectiveness, improved stress tolerance (e.g. against drought, heat, salt, UV, water, cold), reduced ethylene (reduced production and/or inhibition of reception), tillering increase, increase in plant height, bigger leaf blade, less dead basal leaves, stronger tillers, greener leaf color, pigment content, photosynthetic activity, less input needed (such as fertilizers or water), less seeds needed, more productive tillers, earlier flowering, early grain maturity, less plant verse (lodging), increased shoot growth, enhanced plant vigor, increased plant stand and early and better germination.

Improved plant growth preferably refers to improved plant characteristics including: crop yield, more developed root system (improved root growth), improved root size maintenance, improved root effectiveness, tillering increase, increase in plant height, bigger leaf blade, less dead basal leaves, stronger tillers, greener leaf color, photosynthetic activity, more productive tillers, enhanced plant vigor, and increased plant stand.

With regard to the present invention, improved plant growth preferably especially refers to improved plant properties selected from crop yield, more developed root system, improved root growth, improved root size maintenance, improved root effectiveness, tillering increase, and increase in plant height.

The effect of fungal spores on plant growth as defined herein can be determined by comparing plants which are grown under the same environmental conditions, whereby a part of said plants is treated with a composition comprising fungal spores of a certain species and/or strain and another part of said plants is not treated with such fungal spores. Instead, said other part is not treated at all or treated with a placebo (i.e., an application without fungal spores as active ingredients).

Filamentous fungi, as the skilled person is well aware, are distinguished from yeasts because of their tendency to grow in a multicellular, filamentous form under most conditions, in contrast to the primarily unicellular growth of oval or elliptical yeast cells.

Said at least one filamentous fungus may be any fungus exerting a positive effect on plants such as a plant protective or plant growth promoting effect. Accordingly, said fungus may be an entomopathogenic fungus, a nematophagous fungus, a plant growth promoting fungus, a fungus active against plant pathogens such as bacteria or fungal plant pathogens, or a fungus with herbicidal action.

Useful fungal spores, that is spores having one or more of the above properties, may originate from a fungal species selected from the group consisting of Isaria fumosorosea, Penicillium frequentans, Cladosporium cladosporioides, Cladosporium delicatum, Metarhizium spp., Beauveria bassiana, Beauveria brogniartii, Lecanicillium spp., Clonostachys rosea, Nomuraea rileyi, Trichoderma spp., Penicillium bilaii, Coniothyrium minitans and Purpureocillium lilacinum.

NRRL is the abbreviation for the Agricultural Research Service Culture Collection, an international depositary authority for the purposes of deposing microorganism strains under the Budapest treaty on the international recognition of the deposit of microorganisms for the purposes of patent procedure, having the address National Center for Agricultural Utilization Research, Agricultural Research service, U.S. Department of Agriculture, 1815 North university Street, Peroira, Ill. 61604 USA.

ATCC is the abbreviation for the American Type Culture Collection, an international depositary authority for the purposes of deposing microorganism strains under the Budapest treaty on the international recognition of the deposit of microorganisms for the purposes of patent procedure, having the address ATCC Patent Depository, 10801 University Blvd., Manassas, Va. 10110 USA.

Only few fungi with selective herbicidal activity are known, such as F2.1 Phoma macrostroma, in particular strain 94-44B; F2.2 Sclerotinia minor, in particular strain IMI 344141 (e.g. Sarritor by Agrium Advanced Technologies); F2.3 Colletotrichum gloeosporioides, in particular strain ATCC 20358 (e.g. Collego (also known as LockDown) by Agricultural Research Initiatives); F2.4 Stagonospora atriplicis; or F2.5 Fusarium oxysporum, different strains of which are active against different plant species, e.g. the weed Striga hermonthica (Fusarium oxysproum formae specialis strigae).

Exemplary species of plant growth supporting, promoting or stimulating fungi are E2.1 Talaromyces flavus, in particular strain V117b; E2.2 Trichoderma atroviride, in particular strain CNCM I-1237 (e.g. Esquive® WP from Agrauxine, FR), strain no. V08/002387, strain no. NMI No. V08/002388, strain no. NMI No. V08/002389, strain no. NMI No. V08/002390, strain LC52 (e.g. Sentinel from Agrimm Technologies Limited), strain kd (e.g. T-Gro from Andermatt Biocontrol), and/or strain LUI32 (e.g. Tenet from Agrimm Technologies Limited); E2.3 Trichoderma harzianum, in particular strain ITEM 908 or T-22 (e.g. Trianum-P from Koppert); E2.4 Myrothecium verrucaria, in particular strain AARC-0255 (e.g. DiTera™ from Valent Biosciences); E2.5 Penicillium bilaii, in particular strain ATCC 22348 (e.g. JumpStart® from Acceleron BioAg), and/or strain ATCC20851; E2.6 Pythium oligandrum, in particular strains DV74 or M1 (ATCC 38472; e.g. Polyversum from Bioprepraty, CZ); E2.7 Rhizopogon amylopogon (e.g. comprised in Myco-Sol from Helena Chemical Company); E2.8 Rhizopogon fulvigleba (e.g. comprised in Myco-Sol from Helena Chemical Company); E2.9 Trichoderma harzianum, in particular strain TSTh20, strain KD, product Eco-T from Plant Health Products, ZA or strain 1295-22; E2.10 Trichoderma koningii; E2.11 Glomus aggregatum; E2.12 Glomus clarum; E2.13 Glomus deserticola; E2.14 Glomus etunicatum; E2.15 Glomus intraradices; E2.16 Glomus monosporum; E2.17 Glomus mosseae; E2.18 Laccaria bicolor; E2.19 Rhizopogon luteolus; E2.20 Rhizopogon tinctorus; E2.21 Rhizopogon villosulus; E2.22 Scleroderma cepa; E2.23 Suillus granulatus; E2.24 Suillus punctatapies; E2.25 Trichoderma virens, in particular strain GL-21; E2.26 Verticillium albo-atrum (formerly V. dahliae), in particular strain WCS850 (CBS 276.92; e.g. Dutch Trig from Tree Care Innovations); E2.27 Trichoderma asperellum, e.g. strain B35 (Pietr et al., 1993, Zesz. Nauk. A R w Szczecinie 161: 125-137); and E2.28 Purpureocillium lilacinum (previously known as Paecilomyces lilacinus) strain 251 (AGAL 89/030550; e.g. BioAct from Bayer CropScience Biologics GmbH).

In a more preferred embodiment, fungal strains having a beneficial effect on plant growth are selected from Talaromyces flavus, strain VII7b; Trichoderma harzianum, in particular strain KD or strain in product Eco-T from Plant Health Products, SZ; Myrothecium verrucaria, in particular strain AARC-0255 (available as DiTera™ from Valent Biosciences); Penicillium bilaii, strain ATCC 22348; Purpureocillium lilacinum (previously known as Paecilomyces lilacinus) strain 251 (AGAL 89/030550; e.g. BioAct from Bayer CropScience Biologics GmbH); and Trichoderma atroviride strain CNCM I-1237 (e.g. Esquive® WP from Agrauxine, FR), Trichoderma asperellum, e.g. strain B35 (Pietr et al., 1993, Zesz. Nauk. A R w Szczecinie 161: 125-137).

In an even more preferred embodiment, fungal strains having a beneficial effect on plant growth are selected from Purpureocillium lilacinum (previously known as Paecilomyces lilacinus) strain 251 (AGAL 89/030550; e.g. BioAct from Bayer CropScience Biologics GmbH); Penicillium bilaii, strain ATCC 22348 (e.g. JumpStart® from Acceleron BioAg), Talaromyces flavus, strain V117b; Trichoderma atroviride strain CNCM I-1237 (e.g. Esquive® WP from Agrauxine, FR), Trichoderma asperellum, e.g. strain B35 (Pietr et al., 1993, Zesz. Nauk. A R w Szczecinie 161: 125-137).

Bactericidally active fungi are e.g.: A2.2 Aureobasidium pullulans, in particular blastospores of strain DSM14940; A2.3 Aureobasidium pullulans, in particular blastospores of strain DSM 14941; A2.4 Aureobasidium pullulans, in particular mixtures of blastospores of strains DSM14940 and DSM14941; A2.9 Scleroderma citrinum.

Fungi active against fungal pathogens are e.g. B2.1 Coniothyrium minitans, in particular strain CON/M/91-8 (Accession No. DSM-9660; e.g. Contans® from Bayer CropScience Biologics GmbH); B2.2 Metschnikowia fructicola, in particular strain NRRL Y-30752; B2.3 Microsphaeropsis ochrace, in particular strain P130A (ATCC deposit 74412); B2.4 Muscodor albus, in particular strain QST 20799 (Accession No. NRRL 30547); ; B2.5 Trichoderma harzianum rifai, in particular strain KRL-AG2 (also known as strain T-22, /ATCC 208479, e.g. PLANTSHIELD T-22G, Rootshield®, and TurfShield from BioWorks, US) and strain T39 (e.g. Trichodex® from Makhteshim, US); B2.6 Arthrobotrys dactyloides; B2.7 Arthrobotrys oligospora; B2.8 Arthrobotrys superba; B2.9 Aspergillus flavus, in particular strain NRRL 21882 (e.g. Afla-Guard® from Syngenta) or strain AF36 (e.g. AF36 from Arizona Cotton Research and Protection Council, US); B2.10 Gliocladium roseum (also known as Clonostachys rosea f. rosea), in particular strain J1446 (e.g. Prestop® by Lallemand), strain 321U from Adjuvants Plus, strain ACM941 as disclosed in Xue (Efficacy of Clonostachys rosea strain ACM941 and fungicide seed treatments for controlling the root tot complex of field pea, Can Jour Plant Sci 83(3): 519-524), strain IK726 (Jensen D F, et al. Development of a biocontrol agent for plant disease control with special emphasis on the near commercial fungal antagonist Clonostachys rosea strain ‘IK726’; Australas Plant Pathol. 2007;36:95-101), strain 88-710 (WO2007/107000), strain CR7 (WO2015/035504)or strains CRrO, CRM and CRr2 disclosed in WO2017109802; B2.11 Phlebiopsis (or Phlebia or Peniophora) gigantea, in particular strain VRA 1835 (ATCC 90304), strain VRA 1984 (DSM16201), strain VRA 1985 (DSM16202), strain VRA 1986 (DSM16203), strain FOC PG B20/5 (IMI390096), strain FOC PG SP log6 (IMI390097), strain FOC PG SP log5 (IMI390098), strain FOC PG BU3 (IMI390099), strain FOC PG BU4 (IMI390100), strain FOC PG 410.3 (IMI390101), strain FOC PG 97/1062/116/1.1 (IMI390102), strain FOC PG B22/SP1287/3.1 (IMI390103), strain FOC PG SH1 (IMI390104) and/or strain FOC PG B22/SP1190/3.2 (IMI390105) (Phlebiopsis products are e.g. Rotstop® from Verdera and FIN, PG-Agromaster®, PG-Fungler®, PG-IBL®, PG-Poszwald® and Rotex® from e-nema, DE); B2.12 Pythium oligandrum, in particular strain DV74 or M1 (ATCC 38472; e.g. Polyversum from Bioprepraty, CZ); B2.13 Scleroderma citrinum; B2.14 Talaromyces flavus, in particular strain V117b; B2.15 Trichoderma asperellum, in particular strain ICC 012 from Isagro or strain SKT-1 (e.g. ECO-HOPE® from Kumiai Chemical Industry), strain T34 (e.g. ASPERELLO® from Biobest Group NV and T34 BIOCONTROL® by Biocontrol Technologies S.L., ES), or strain B35 (Pietr et al., 1993, Zesz. Nauk. A R w Szczecinie 161: 125-137); B2.16 Trichoderma atroviride, in particular strain CNCM I-1237 (e.g. Esquive® WP from Agrauxine, FR), strain SC1 described in International Application No. PCT/IT2008/000196), strain 77B (T77 from Andermatt Biocontrol), strain no. V08/002387, strain NMI no. V08/002388, strain NMI no. V08/002389, strain NMI no. V08/002390, strain LC52 (e.g. Sentinel from Agrimm Technologies Limited), strain LUI32 (e.g. Tenet by Agrimm Technologies Limited), strain ATCC 20476 (IMI 206040), strain T11 (IMI352941/CECT20498), strain SKT-1 (FERM P-16510), strain SKT-2 (FERM P-16511), strain SKT-3 (FERM P-17021); B2.17 Trichoderma harmatum; ; B2.18 Trichoderma harzianum, in particular, strain KD, strain ITEM 908 (e.g. Trianum-P from Koppert), strain TH35 (e.g. Root-Pro by Mycontrol), strain DB 103 (e.g. T-Gro 7456 by Dagutat Biolab); B2.19 Trichoderma virens (also known as Gliocladium virens), in particular strain GL-21 (e.g. SoilGard by Certis, US); B2.20 Trichoderma viride, in particular strain TV1 (e.g. Trianum-P by Koppert); B2.21 Ampelomyces quisqualis, in particular strain AQ 10 (e.g. AQ 10® by CBC Europe, Italy); B2.22 Arkansas fungus 18, ARF; B2.23 Aureobasidium pullulans, in particular blastospores of strain DSM14940, blastospores of strain DSM 14941 or mixtures of blastospores of strains DSM14940 and DSM 14941 (e.g. Botector® by bio-ferm, CH); B2.24 Chaetomium cupreum (e.g. BIOKUPRUM™ by AgriLife); B2.25 Chaetomium globosum (e.g. Rivadiom by Rivale); B2.26 Cladosporium cladosporioides, in particular strain H39 (by Stichting Dienst Landbouwkundig Onderzoek); B2.27 Dactylaria candida; B2.28 Dilophosphora alopecuri (e.g. Twist Fungus); B2.29 Fusarium oxysporum, in particular strain Fo47 (e.g. Fusaclean by Natural Plant Protection); B2.30 Gliocladium catenulatum (Synonym: Clonostachys rosea f. catenulate), in particular strain J1446 (e.g. Prestop® by Lallemand); B2.31 Lecanicillium lecanii (formerly known as Verticillium lecanii), in particular conidia of strain KV01 (e.g. Vertalec® by Koppert/Arysta); B2.32 Penicillium vermiculatum; ; B2.33 Trichoderma gamsii (formerly T. viride), in particular strain ICC080 (IMI CC 392151 CABI, e.g. BioDerma by AGROBIOSOL DE MEXICO, S.A. DE C.V.); B2.34 Trichoderma polysporum, in particular strain IMI 206039 (e.g. Binab TF WP by BINAB Bio-Innovation AB, Sweden); B2.35 Trichoderma stromaticum (e.g. Tricovab by Ceplac, Brazil); B2.36 Tsukamurella paurometabola, in particular strain C-924 (e.g. HeberNem®); B2.37 Ulocladium oudemansii, in particular strain HRU3 (e.g. Botry-Zen® by Botry-Zen Ltd, NZ); B2.38 Verticillium albo-atrum (formerly V. dahliae), in particular strain WCS850 (CBS 276.92; e.g. Dutch Trig by Tree Care Innovations); B2.39 Muscodor roseus, in particular strain A3-5 (Accession No. NRRL 30548); B2.40 Verticillium chlamydosporium; B2.41 mixtures of Trichoderma asperellum strain ICC 012 and Trichoderma gamsii strain ICC 080 (product known as e.g. BIO-TAM™ from Bayer CropScience LP, US), B2.42 Simplicillium lanosoniveum; Trichoderma asperelloides JM41R (Accession No. NRRL B-50759) (TRICHO PLUS® from BASF SE); and B2.43 Trichoderma fertile (e.g. product TrichoPlus from BASF).

In a preferred embodiment, the biological control agent having fungicidal activity is selected from Coniothyrium minitans strain CON/M/91-8 (Accession No. DSM-9660; e.g. Contans® from Bayer CropScience Biologics GmbH); Talaromyces flavus strain V117b; Trichoderma atroviride strain CNCM I-1237 (e.g. Esquive® WP from Agrauxine, FR); Gliocladium catenulatum (Synonym: Clonostachys rosea f. catenulate) strain J1446 (e.g. Prestop® by Lallemand); Trichoderma asperellum strain B35 (Pietr et al., 1993, Zesz. Nauk. A R w Szczecinie 161: 125-137); Metschnikowia fructicola strain NRRL Y-30752; Gliocladium roseum (also known as Clonostachys rosea f. rosea) strain 321U from Adjuvants Plus, strain ACM941 as disclosed in Xue (Efficacy of Clonostachys rosea strain ACM941 and fungicide seed treatments for controlling the root tot complex of field pea, Can Jour Plant Sci 83(3): 519-524), strain IK726 (Jensen D F, et al. Development of a biocontrol agent for plant disease control with special emphasis on the near commercial fungal antagonist Clonostachys rosea strain ‘IK726’; Australas Plant Pathol. 2007; 36:95-101); Trichoderma asperellum strain SKT-1, having Accession No. FERM P-16510 (e.g., ECO-HOPE® from Kumiai Chemical Industry); Trichoderma asperellum T34 (ASPERELLO® from Biobest Group NV and T34 BIOCONTROL® by Biocontrol Technologies S.L., ES); Trichoderma asperelloides JM41R (Accession No. NRRL B-50759) (TRICHO PLUS® from BASF SE); Trichoderma asperellum strain ICC 012 (Isagro); Trichoderma atroviride strain SC1, having Accession No. CBS 122089, WO 2009/116106 and U.S. Pat. No. 8,431,120 (from Bi-PA); Trichoderma atroviride strain 77B (T77 from Andermatt Biocontrol); Trichoderma atroviride strain LU132 (e.g. Sentinel from Agrimm Technologies Limited); Trichoderma harzianum strain T-22 (e.g. Trianum-P from Andermatt Biocontrol or Koppert); Trichoderma virens (also known as Gliocladium virens), in particular strain GL-21 (e.g. SoilGard by Certis, US); and Trichoderma harzianum strain Cepa Simb-T5 (from Simbiose Agro).

In a more preferred embodiment, the fungal species having fungicidal activity is selected from Coniothyrium minitans strain CON/M/91-8 (Accession No. DSM-9660; e.g. Contans® from Bayer Crop Science Biologics GmbH); Talaromyces flavus strain V117b; Trichoderma atroviride strain CNCM I-1237 (e.g. Esquive® WP from Agrauxine, FR); Gliocladium catenulatum (Synonym: Clonostachys rosea f. catenulate) strain J1446 (e.g. Prestop® by Lallemand); Trichoderma asperellum strain B35 (Pietr et al., 1993, Zesz. Nauk. A R w Szczecinie 161: 125-137) and Gliocladium catenulatum (strain J1446 (e.g. Prestop® by Lallemand).

Within fungicidally active and/or plant growth promoting fungi, the genus Trichoderma spp., or their respective teleomorphs, Hypocrea spp. are preferred. Preferably said fungal strains belong to the species Tichoderma atroviride, Trichoderma asperellum, Trichoderma harzianum, Trichoderma viride, Trichoderma virens Trichoderma koningii, Trichoderma hamatum, Trichoderma gamsii, Trichoderma stromaticum, Trichoderma fertile, Trichoderma longibrachiatum or Trichoderma polysporum. Exemplary strains, belonging to said species which are preferred are Trichoderma atroviride strain NMI no. V08/002387 (described in U.S. Pat. No. 8,394,623B2), strain NMI no. V08/002388, strain NMI no. V08/002389, strain NMI no. V08/002390, strain LC52 (e.g. Sentinel or Tenet from Agrimm Technologies Limited), strain CNCM I-1237 (e.g. Esquive from Agrauxine, France), strain SC1 (e.g. Vintec from Bi-PA or Belchim, described in International Application No. PCT/IT2008/000196), strain B77 (e.g. T77 from Andermatt Biocontrol or Eco-77 from Plant Health Products), strain LUI32 (e.g. Tenet from Agrimm Technologies Limited), strain IMI 206040/ATCC 20476 (e.g. Binab TF WP from BINAB Bio-Innovation AB, Sweden), strain T11/IMI 352941/CECT 20498 (e.g. Tusal from Certis), strain SKT-1/FERM P-16510 (e.g. ECO-HOPE from Kumiai Chemical Industry Co), strain SKT-2/FERM P-16511, strain SKT-3/FERM P-17021, strain MUCL45632 (e.g. Tandem from Italpollina), strain WW10TC4/ATCC PTA 9707 (described in CA2751694A1), strain RR17Bc/ATCC PTA 9708, strain F11 Bab/ATCC PTA 9709; strain TF280 (described in CN107034146A), strain OB-1/KCCM 11173P (described in WO2012124863A1); Trichoderma harzianum strain KRL-AG2/ITEM 908/T-22/ATCC 20847 (e.g.

Trianum-P from Koppert or PlantShield from BioWorks or Tricho D WP from Orius Biotecnologica), strain TH35 (e.g. Root-Pro from Mycontrol), strain T-39 (e.g. TRICHODEX and TRICHODERMA 2000 from Mycontrol), strain DB 103 (e.g. T-Gro 7456 from Dagutat Biolab, South Africa), strain DB 104 (e.g. Romulus from Dagutat Biolab, South Africa), strain TSTh20/ATCC PTA-10317 (described in Application EP2478090A1), strain ESALQ 1306 (e.g. Trichodermil from Koppert), Rifai strain KRL-AG2 (e.g. BW240 WP from BioWorks), strain T78 (e.g. OffYouGrow Tric from Microgaia Biotech), strain from Trichopel (Agrimm Technologies), strain RR17Bc/ATCC PTA 9708 (described in CA2751694A1), strain ThLm1/NRRL 50846 (described in US20150033420A1), strain IBLF 006 (e.g. Ecotrich W P and Predatox S C from Ballagro Agro Tecnologia Ltda., Brazil), strain DSM 14944 (e.g. Agroguard W G and Foliguard from Live Systems Technology S.A, Colombia), strain 21 (e.g. Rootgard from Juanco SPS Ltd., Kenya), strain SF (e.g. Bio-Tricho from Agro-Organics, South Africa), strain IIHR-Th-2 (e.g. Ecosom-TH from Agri Life, India), strain MTCC5530 (described in US20120015806A1); Trichoderma virens (also known as Gliocladium virens) strain GL-21 (e.g. SoilGard by Certis, USA) strain G1-21, strain G1-3/ATCC 58678 (e.g. QuickRoots from Novozymes), strain DSM25764, strain G-41 (e.g. RootShieldPlus from BioWorks); Trichoderma viride strain TV1/MUCL 43093 (e.g. Virisan from Isagro), strain MTCC5532 (described in US20120015806A1), strain NRRL B-50520 (described in CN104203871A); Trichoderma polysporum strain IMI 206039/ATCC 20475/T-75 (e.g. Binab TF WP from BINAB Bio-Innovation AB, Sweden); Trichoderma stromaticum strain Ceplac 3550/ALF 64 (Tricovab from Ceplac, Brazil); Trichoderma asperellum strain kd (e.g. T-Gro from Andermatt Biocontrol or ECO-T from Plant Health Products), strain ICC 012/IMI 392716 (e.g. BIO-TAM and REMEDIER WP from Isagro Ricerca), strain B35 (Pietr et al., 1993, Zesz. Nauk. A R w Szczecinie 161: 125-137), strain BV10 (e.g. Tricho-Turbo from Biovalens), strain T34 (e.g. Asperello T34 Biocontrol from Biobest), strain T25/IMI 296237/CECT 20178 (e.g. Tusal from Certis), strain SKT-1 (e.g. Ecohope from Kumiai Chemical Industry Co.), strain URM 5911/SF04 (e.g. Quality WG from Laboratorio de BioControle Farroupilha Ltda, Patos de Minas-MG, Brazil), strain H22 (e.g. TRICHOTECH WP from Dudutech); Trichoderma gamsii strain ICC 080 (e.g. BIO-TAM and REMEDIER WP from Isagro Ricerca), strain NRRL B-50520 (described in W02017192117A1); Trichoderma koningii strain SC164; Trichoderma hamatum strain TH382/ATCC 20765 (e.g. Floragard from Sellew Associates); Trichoderma fertile strain JM41R (e.g. TrichoPlus from BASF); Trichoderma longibrachiatum strain Mk1/KV966 (described in WO2015126256A1). The species Trichoderma viride and Trichoderma atroviride, are especially preferred. Even more preferred strain of these species are Trichoderma atroviride strain CNCM I-1237 (e.g. Esquive® WP from Agrauxine, FR); Trichoderma atroviride strain SC1, having Accession No. CBS 122089, WO 2009/116106 and U.S. Pat. No. 8,431,120 (from Bi-PA); Trichoderma atroviride strain 77B (T77 from Andermatt Biocontrol); Trichoderma atroviride strain LU132 (e.g. Sentinel from Agrimm Technologies Limited); Trichoderma asperellum strain B35 (Pietr et al., 1993, Zesz. Nauk. A R w Szczecinie 161: 125-137). Particularly preferred are Trichoderma atroviride strain CNCM I-1237 and Trichoderma asperellum strain B35.

Said fungal microorganism may be an entomopathogenic fungus.

Fungi active against insects and mites (entomopathogenic fungi) include C2.1 Muscodor albus, in particular strain QST 20799 (Accession No. NRRL 30547); C2.2 Muscodor roseus in particular strain A3-5 (Accession No. NRRL 30548); C2.3 Beauveria bassiana, in particular strain ATCC 74040 (e.g. Naturalis® from Intrachem Bio Italia); strain GHA (Accession No. ATCC74250; e.g. BOTANIGUARD® ES and MYCONTROL-O® from Laverlam International Corporation); strain ATP02 (Accession No. DSM 24665); strain PPRI 5339 (e.g. BroadBand™ from BASF); strain PPRI 7315, strain R444 (e.g. Bb-Protec from Andermatt Biocontrol), strains IL197, IL12, IL236, IL10, IL131, IL116 (all referenced in Jaronski, 2007. Use of Entomopathogenic Fungi in Biological Pest Management, 2007: ISBN: 978-81-308-0192-6), strain Bv025 (see e.g. Garcia et al. 2006. Manejo Integrado de Plagas y Agroecologia (Costa Rica) No. 77); strain BaGPK; strain ICPE 279, strain CG 716 (e.g. BoveMax® from Novozymes); C2.4 Hirsutella citriformis; C2.5 Hirsutella thompsonii (e.g. Mycohit and ABTEC from Agro Bio-tech Research Centre, IN); C2.6 Lecanicillium lecanii (formerly known as Verticillium lecanii), in particular conidia of strain KV01 (e.g. Mycotal® and Vertalec® from Koppert/Arysta), strain DAOM198499 or strain DAOM216596; C2.9 Lecanicillium muscarium (formerly Verticillium lecanii), in particular strain VE 6/CABI(=IMI) 268317/CBS102071/ARSEF5128 (e.g. Mycotal from Koppert); C2.10 Metarhizium anisopliae var acridum, e.g. ARSEF324 from GreenGuard by Becker Underwood, US or isolate IMI 330189 (ARSEF7486; e.g. Green Muscle by Biological Control Products); C2.11 Metarhizium brunneum, e.g. strain Cb 15 (e.g. ATTRACAP® from BIOCARE); C2.12 Metarhizium anisopliae, e.g. strain ESALQ 1037 (e.g. from Metarril® SP Organic), strain E-9 (e.g. from Metarril® SP Organic), strain M206077, strain C4-B (NRRL 30905), strain ESC1, strain 15013-1 (NRRL 67073), strain 3213-1 (NRRL 67074), strain C20091, strain C20092, strain F52 (DSM3884/ATCC 90448; e.g. BIO 1020 by Bayer CropScience and also e.g. Met52 by Novozymes) or strain ICIPE 78; C2.15 Metarhizium robertsii 23013-3 (NRRL 67075); C2.13 Nomuraea rileyi; C2.14 Paecilomyces fumosoroseus (new: Isaria fumosorosea), in particular strains Apopka 97 (available as PreFeRal from Certis, USA), Fe9901 (available as NoFly from Natural industries, USA), ARSEF 3581, ARSEF 3302, ARSEF 2679 (ARS Collection of Entomopathogenic Fungal Cultures, Ithaca, USA), IfB01 (China Center for Type Culture Collection CCTCC M2012400), ESALQ1296, ESALQ1364, ESALQ1409 (ESALQ: University of São Paulo (Piracicaba, SP, Brazil)), CG1228 (EMBRAPA Genetic Resources and Biotechnology (Brasilia, DF, Brazil)), KCH J2 (Dymarska et al., 2017; PLoS one 12(10)): e0184885), HIB-19, HIB-23, HIB-29, HIB-30 (Gandarilla-Pacheco et al., 2018; Rev Argent Microbiol 50: 81-89), CHE-CNRCB 304, EH-511/3 (Flores-Villegas et al., 2016; Parasites & Vectors 2016 9:176 doi: 10.1186/s13071-016-1453-1), CHE-CNRCB 303, CHE-CNRCB 305, CHE-CNRCB 307 (Gallou et al., 2016; fungal biology 120 (2016) 414-423), EH-506/3, EH-503/3, EH-520/3, PFCAM, MBP, PSMB1 (National Center for Biololgical Control, Mexico; Castellanos-Moguel et al., 2013; Revista Mexicana De Micologia 38: 23-33, 2013), RCEF3304 (Meng et al., 2015; Genet Mol Biol. 2015 July-September; 38(3): 381-389), PF01-N10 (CCTCC No. M207088), CCM 8367 (Czech Collection of Microorganisms, Brno), SFP-198 (Kim et al., 2010; Wiley Online: DOI 10.1002/ps.2020), K3 (Yanagawa et al., 2015; J Chem Ecol. 2015; 41(12): 118-1126), CLO 55 (Ansari Ali et al., 2011; PLoS One. 2011; 6(1): e16108. DOI: 10.1371/journal.pone.0016108), IfTS01, IfTS02, IfTS07 (Dong et al. 2016/PLoS ONE 11(5): e0156087. doi:10.1371/journal.pone.0156087), P1 (Sun Agro Biotech Research Centre, India), If-02, If-2.3, If-03 (Farooq and Freed, 2016; DOI: 10.1016/j.bjm.2016.06.002), Ifr AsC (Meyer et al., 2008; J. Invertebr. Pathol. 99:96-102. 10.1016/jjip.2008.03.007), PC-013 (DSMZ 26931), P43A, PCC (Carrillo-Pérez et al., 2012; DOI 10.1007/s11274-012-1184-1), Pf04, Pf59, Pf109 (KimJun et al., 2013; Mycobiology 2013 December; 41(4): 221-224), FG340 (Han et al., 2014; DOI: 10.5941/MYCO.2014.42.4.385), Pfrl, Pfr8, Pfr9, Pfr10, Pfr11, Pfr12 (Angel-Sahagún et al., 2005; Journal of Insect Science), Ifr531 (Daniel and Wyss, 2009; DOI 10.1111/j.1439-0418.2009.01410.x), IF-1106 (Insect Ecology and Biocontrol Laboratory, Shanxi Agricultural University), I9602, I7284 (Hussain et al. 2016, DOI:10.3390/ijms17091518), I03011 (U.S. Pat. No. 4,618,578), CNRCB1 (Centro Nacional de Referencia de Control Biologico (CNRCB), Colima, Mexico), SCAU-IFCF01 (Nian et al., 2015; DOI: 10.1002/ps.3977), PF01-N4 (Engineering Research Center of Biological Control, SCAU, Guangzhou, P. R. China) Pfr-612 (Institute of Biotechnology (IB-FCB-UANL), Mexico), Pf-Tim, Pf-Tiz, Pf-Hal, Pf-Tic (Chan-Cupul et al. 2013, DOI: 10.5897/AJMR12.493); C2.15 Aschersonia aleyrodis; C2.16 Beauveria brongniartii (e.g. Beaupro from Andermatt Biocontrol AG); C2.17 Conidiobolus obscurus; C2.18 Entomophthora virulenta (e.g. Vektor from Ecomic); C2.19 Lagenidium giganteum; C2.20 Metarhizium flavoviride; C2.21 Mucor haemelis (e.g. BioAvard from Indore Biotech Inputs & Research); C2.22 Pandora delphacis; C2.23 Sporothrix insectorum (e.g. Sporothrix Es from Biocerto, BR); C2.24 Zoophtora radicans.

In a more preferred embodiment, fungal strains having an insecticidal effect are selected from C2.3 Beauveria bassiana, in particular strain ATCC 74040; strain GHA (Accession No. ATCC74250); strain ATP02 (Accession No. DSM 24665); strain PPRI 5339; strain PPRI 7315, strain R444, strains IL197, IL12, IL236, IL10, IL131, IL116; strain BaGPK; strain ICPE 279, strain CG 716; C2.6 Lecanicillium lecanii (formerly known as Verticillium lecanii), in particular conidia of strain KV01, strain DAOM198499 or strain DAOM216596; C2.9 Lecanicillium muscarium (formerly Verticillium lecanii), in particular strain VE 6/CABI(=IMI) 268317/CBS102071/ARSEF5128; C2.10 Metarhizium anisopliae var acridum, e.g. ARSEF324 or isolate IMI 330189 (ARSEF7486); C2.11 Metarhizium brunneum, e.g. strain Cb 15; C2.12 Metarhizium anisopliae, e.g. strain ESALQ 1037, strain E-9, strain M206077, strain C4-B (NRRL 30905), strain ESC1, strain 15013-1 (NRRL 67073), strain 3213-1 (NRRL 67074), strain C20091, strain C20092, strain F52 (DSM3884/ATCC 90448) or strain ICIPE 78; C2.14 Paecilomyces fumosoroseus (new: Isaria fumosorosea), in particular strains Apopka 97, Fe9901, ARSEF 3581, ARSEF 3302, ARSEF 2679, IfB01 (China Center for Type Culture Collection CCTCC M2012400), ESALQ1296, ESALQ1364, ESALQ1409, CG1228, KCH J2, HIB-19, HIB-23, HIB-29, HIB-30, CHE-CNRCB 304, EH-511/3, CHE-CNRCB 303, CHE-CNRCB 305, CHE-CNRCB 307, EH-506/3, EH-503/3, EH-520/3, PFCAM, MBP, PSMB1, RCEF3304, PF01-N10 (CCTCC No. M207088), CCM 8367, SFP-198, K3, CLO 55, IfTS01, IfTS02, IfTS07, P1, If-02, If-2.3, If-03, Ifr AsC, PC-013 (DSMZ 26931), P43A, PCC, Pf04, Pf59, Pf109, FG340, Pfr1, Pfr8, Pfr9, Pfr10, Pfr11, Pfr12, Ifr531, IF-1106, 19602, 17284, 103011 (U.S. Pat. No. 4,618,578), CNRCB1, SCAU-IFCF01, PF01-N4, Pfr-612, Pf-Tim, Pf-Tiz, Pf-Hal and Pf-Tic.; and C2.16 Beauveria brongniartii (e.g. Beaupro from Andermatt Biocontrol AG).

In an even more preferred embodiment, fungal strains having an insecticidal effect are selected from C2.3 Beauveria bassiana, in particular strain ATCC 74040; strain GHA (Accession No. ATCC74250); strain ATP02 (Accession No. DSM 24665); strain PPRI 5339; strain PPRI 7315, strain R444, strains IL197, IL12, IL236, IL10, IL131, IL116; strain BaGPK; strain ICPE 279, strain CG 716; C2.6 Lecanicillium lecanii (formerly known as Verticillium lecanii), in particular conidia of strain KV01, strain DAOM198499 or strain DAOM216596; C2.9 Lecanicillium muscarium (formerly Verticillium lecanii), in particular strain VE 6/CABI(=IMI) 268317/CBS102071/ARSEF5128; C2.10 Metarhizium anisopliae var acridum, e.g. ARSEF324 or isolate IMI 330189 (ARSEF7486); C2.11 Metarhizium brunneum, e.g. strain Cb 15; C2.12 Metarhizium anisopliae, e.g. strain ESALQ 1037, strain E-9, strain M206077, strain C4-B (NRRL 30905), strain ESC1, strain 15013-1 (NRRL 67073), strain 3213-1 (NRRL 67074), strain C20091, strain C20092, strain F52 (DSM3884/ATCC 90448) or strain ICIPE 78; C2.14 Paecilomyces fumosoroseus (new: Isaria fumosorosea), in particular strains Apopka 97, Fe9901, ARSEF 3581, ARSEF 3302, ARSEF 2679, IfB01 (China Center for Type Culture Collection CCTCC M2012400), ESALQ1296, ESALQ1364, ESALQ1409, CG1228, KCH J2, HIB-19, HIB-23, HIB-29, HIB-30, CHE-CNRCB 304, EH-511/3, CHE-CNRCB 303, CHE-CNRCB 305, CHE-CNRCB 307, EH-506/3, EH-503/3, EH-520/3, PFCAM, MBP, PSMB1, RCEF3304, PF01-N10 (CCTCC No. M207088), CCM 8367, SFP-198, K3, CLO 55, IfTS01, IfTS02, IfTS07, P1, If-02, If-2.3, If-03, Ifr AsC, PC-013 (DSMZ 26931), P43A, PCC, Pf04, Pf59, Pf109, FG340, Pfr1, , Pfr8, Pfr9, Pfr10, Pfr11, Pfr12, Ifr531, IF-1106, 19602, 17284 , 103011 (U.S. Pat. No. 4,618,578), CNRCB1, SCAU-IFCF01, PF01-N4, Pfr-612, Pf-Tim, Pf-Tiz, Pf-Hal and Pf-Tic and C2.16 Beauveria brongniartii (e.g. Beaupro from Andermatt Biocontrol AG).

It is even more preferred that said fungal microorganism is a strain of the species Isaria fumosorosea. Preferred strains of Isaria fumosorosea are selected from the group consisting of Apopka 97, Fe9901, ARSEF 3581, ARSEF 3302, ARSEF 2679, IfB01 (China Center for Type Culture Collection CCTCC M2012400), ESALQ1296, ESALQ1364, ESALQ1409, CG1228, KCH J2, HIB-19, HIB-23, HIB-29, HIB-30, CHE-CNRCB 304, EH-511/3, CHE-CNRCB 303, CHE-CNRCB 305, CHE-CNRCB 307, EH-506/3, EH-503/3, EH-520/3, PFCAM, MBP, PSMB1, RCEF3304, PF01-N10 (CCTCC No. M207088), CCM 8367, SFP-198, K3, CLO 55, IfTS01, IfTS02, IfTS07, P1, If-02, If-2.3, If-03, Ifr AsC, PC-013 (DSMZ 26931), P43A, PCC, Pf04, Pf59, Pf109, FG340, Pfr1, Pfr8, Pfr9, Pfr10, Pfr11, , Pfr12, Ifr531, IF-1106, 19602, 17284, 103011 (U.S. Pat. No. 4,618,578), CNRCB1, SCAU-IFCF01, PF01-N4, Pfr-612, Pf-Tim, Pf-Tiz, Pf-Hal, Pf-Tic.

It is most preferred that said Isaria fumosorosea strain is selected from Apopka 97 and Fe9901. A particularly preferred strain is APOPKA97. As can be seen in the examples, the method according to the invention was particularly effective for this strain in a formulation based on Tween 20 as surfactant.

Also particularly preferred are entomopathogenic fungi of the genus Metarhizium spp. The genus Metahrizium comprises several species some of which have recently been re-classified (for an overview, see Bischoff et al., 2009; Mycologia 101 (4): 512-530). Members of the genus Metarhizium comprise M. pingshaense, M. anisopliae, M. robertsii, M. brunneum (these four are also referred to as Metarhizium anisopliae complex), M. acridum, M. majus, M. guizouense, M. Lepidiotae, M. Globosum and M. rileyi (previously known as Nomuraea rileyi). Of these, M. anisopliae, M. robertsii, M. brunneum, M. acridum and M. rileyi are even more preferred, whereas those of M. brunneum are most preferred.

Exemplary strains belonging to Metarhizium spp. which are also especially preferred are Metarhizium acridum ARSEF324 (product GreenGuard by BASF) or isolate IMI 330189 (ARSEF7486; e.g. Green Muscle by Biological Control Products); Metarhizium brunneum strain Cb 15 (e.g. ATTRACAP® from BIOCARE), or strain F52 (DSM3884/ATCC 90448; e.g. BIO 1020 by Bayer CropScience and also e.g. Met52 by Novozymes); Metarhizium anisopliae complex strains strain ESALQ 1037 or strain ESALQ E-9 (both from Metarril® WP Organic), strain M206077, strain C4-B (NRRL 30905), strain ESC1, strain 15013-1 (NRRL 67073), strain 3213-1 (NRRL 67074), strain C20091, strain C20092, or strain ICIPE 78. Most preferred is isolate F52 (a.k.a. Met52) which primarily infects beetle larvae and which was originally developed for control of Otiorhynchus sulcatus. and ARSEF324 which is commercially used in locust control. Commercial products based on the F52 isolate are subcultures of the individual isolate F52 and are represented in several culture collections including: Julius Kühn-Institute for Biological Control (previously the BBA), Darmstadt, Germany: [as M.a. 43]; HRI, UK: [275-86 (acronyms V275 or KVL 275)]; KVL Denmark [KVL 99-112 (Ma 275 or V 275)]; Bayer, Germany [DSM 3884]; ATCC, USA [ATCC 90448]; USDA, Ithaca, USA [ARSEF 1095]. Granular and emulsifiable concentrate formulations based on this isolate have been developed by several companies and registered in the EU and North America (US and Canada) for use against black vine weevil in nursery omamentals and soft fruit, other Coleoptera, western flower thrips in greenhouse ornamentals and chinch bugs in turf.

Beauveria bassiana is mass-produced and used to manage a wide variety of insect pests including whiteflies, thrips, aphids and weevils. Preferred strains of Beauveria bassiana include strain ATCC 74040; strain GHA (Accession No. ATCC74250); strain ATP02 (Accession No. DSM 24665); strain PPRI 5339; strain PPRI 7315, strains IL197, IL12, IL236, IL10, IL131, IL116, strain Bv025; strain BaGPK; strain ICPE 279, strain CG 716; ESALQPL63, ESALQ447 and ESALQ1432, CG1229, IMI389521, NPP111B005, Bb-147. It is most preferred that Beauveria bassiana strains include strain ATCC 74040 and strain GHA (Accession No. ATCC74250).

Said fungal species may also be a nematicidally active fungus.

Nematicidally active fungal species include D2.1 Muscodor albus, in particular strain QST 20799 (Accession No. NRRL 30547); D2.2 Muscodor roseus, in particular strain A3-5 (Accession No. NRRL 30548); D2.3 Purpureocillium lilacinum (previously known as Paecilomyces lilacinus), in particular P. lilacinum strain 251 (AGAL 89/030550; e.g. BioAct from Bayer CropScience Biologics GmbH); D2.4 Trichoderma koningii; D2.5 Harposporium anguillullae; D2.6 Hirsutella minnesotensis; D2.7 Monacrosporium cionopagum; D2.8 Monacrosporium psychrophilum; D2.9 Myrothecium verrucaria, in particular strain AARC-0255 (e.g. DiTera™ by Valent Biosciences); D2.10 Paecilomyces variotii, strain Q-09 (e.g. Nemaquim® from Quimia, MX); D2.11 Stagonospora phaseoli (e.g. from Syngenta); D2.12 Trichoderma lignorum, in particular strain TL-0601 (e.g. Mycotric from Futureco Bioscience, ES); D2.13 Fusarium solani, strain Fs5; D2.14 Hirsutella rhossiliensis; D2.15 Monacrosporium drechsleri; D2.16 Monacrosporium gephyropagum; D2.17 Nematoctonus geogenius; D2.18 Nematoctonus leiosporus; D2.19 Neocosmospora vasinfecta; D2.20 Paraglomus sp, in particular Paraglomus brasilianum; D2.21 Pochonia chlamydosporia (also known as Vercillium chlamydosporium), in particular var. catenulata (IMI SD 187; e.g. KlamiC from The National Center of Animal and Plant Health (CENSA), CU); D2.22 Stagonospora heteroderae; D2.23 Meristacrum asterospermum, D2.24 Duddingtonia flagrans.

In a more preferred embodiment, fungal strains with nematicidal effect are selected from Purpureocillium lilacinum, in particular spores of P. lilacinum strain 251 (AGAL 89/030550) (available as BioAct from Bayer CropScience Biologics GmbH); Harposporium anguillullae; Hirsutella minnesotensis; Monacrosporium cionopagum; Monacrosporium psychrophilum; Myrothecium verrucaria, strain AARC-0255 (available as DiTera™ by Valent Biosciences); Paecilomyces variotii; Stagonospora phaseoli (commercially available from Syngenta); and Duddingtonia flagrans.

In an even more preferred embodiment, fungal strains with nematicidal effect are selected from Purpureocillium lilacinum, in particular spores of P. Lilacinum strain 251 (AGAL 89/030550) (available as BioAct from Bayer CropScience Biologics GmbH); and Duddingtonia flagrans.

It is even more preferred that said fungal microorganim is Purpureocillium lilacinum. A number of Purpureocillium lilacinum strains have been described for use as a biological control agent. Such strains include strain 251 in the products BioAct, MeloCon and NemOut produced by Bayer CropScience Biologics GmbH, a strain 580 in the product Biostat WP (ATCC no. 38740) produced by Laverlam, a strain in the product Bio-Nematon produced by the company T. Stanes and Company Ltd., a strain in the product Mysis produced by the company Varsha Bioscience and Technology India Pvt Ltd., one in the product Bioiconema available from Nico Orgo Maures, India, one in the product Nemat, available from Ballagro Agro Tecnologia Ltda, Brazil and one in the product Spectrum Pae L available from Promotora Tecnica Industrial, S.A. DE C.V., Mexico.

It is most preferred that said Purpureocillium lilacinum is Purpureocillium lilacinum strain 251 as described in WO1991/002051 or a mutant thereof having all identifying characteristics of the respective strain. In this embodiment, said ratio between liquid composition and water-based liquid is preferably between 50:1 and 1:5. As can be seen in the examples, the method of the invention works particularly well for this strain if formulated in a polyether-modified trisiloxane, optionally with added fumed or precipitated silica.

Although specific fungal propagules such as microsclerotia (see e.g. Jackson and Jaronski (2009). Production of microsclerotia of the fungal entomopathogen Metarhizium anisopliae and their potential for use as a biocontrol agent for soil-inhabiting insects; Mycological Research 113, pp. 842-850) may be produced by liquid fermentation techniques, it is preferred that the fungal spores are produced by solid-state fermentation. Solid-state fermentation techniques are well known in the art (for an overview see Gowthaman et al., 2001. Appl Mycol Biotechnol (1), p. 305-352).

After fermentation, the fungal spores may be separated from the substrate. The substrate populated with the fungal spores may be dried before any separation step or after separation. The microorganism or fungal spores may be dried via e. g. freeze-drying, vacuum drying or spray drying after separation. Methods for preparing dried spores are well known in the art and include fluidized bed drying, spray drying, vacuum drying and lyophilization. Conidia may be dried in 2 steps: For conidia produced by solid-state fermentation first the conidia covered culture substrate may be dried before harvesting the conidia from the dried culture substrate thereby obtaining a pure conidia powder. Then the conidia powder is dried further using vacuum drying or lyophilization before storing or formulating it. Alternatively, conidia may be wet-harvested and dried afterwards.

After suspending the liquid composition comprising fungal spores in an agriculturally acceptable water-based liquid the resulting mixture may be applied in agriculture in any desired manner, such as in the form of a seed coating, soil drench, and/or directly in-furrow and/or as a foliar spray and applied either pre-emergence, post-emergence or both. In other words, the resulting mixture can be applied to the seed, the plant or to harvested fruits and vegetables or to the soil wherein the plant is growing or wherein it is desired to grow (plant's locus of growth). Customary application methods include for example dipping, spraying, atomizing, irrigating, evaporating, dusting, fogging, broadcasting, foaming, painting, spreading-on, watering (drenching) and drip irrigating.

All plants and plant parts can be treated in accordance with the invention. Here, plants are to be understood to mean all plants and plant parts such as wanted and unwanted wild plants or crop plants (including naturally occurring crop plants),

Plants which can be treated in accordance with the invention include the following main crop plants: maize, soya bean, alfalfa, cotton, sunflower, Brassica oil seeds such as Brassica napus (e.g. canola, rapeseed), Brassica rapa, B. juncea (e.g. (field) mustard) and Brassica carinata, Arecaceae sp. (e.g. oilpalm, coconut), rice, wheat, sugar beet, sugar cane, oats, rye, barley, millet and sorghum, triticale, flax, nuts, grapes and vine and various fruit and vegetables from various botanic taxa, e.g. Rosaceae sp. (e.g. pome fruits such as apples and pears, but also stone fruits such as apricots, cherries, almonds, plums and peaches, and berry fruits such as strawberries, raspberries, red and black currant and gooseberry), Ribesioidae sp., Juglandaceae sp., Betulaceae sp., Anacardiaceae sp., Fagaceae sp., Moraceae sp., Oleaceae sp. (e.g. olive tree), Actinidaceae sp., Lauraceae sp. (e.g. avocado, cinnamon, camphor), Musaceae sp. (e.g. banana trees and plantations), Rubiaceae sp. (e.g. coffee), Theaceae sp. (e.g. tea), Sterculiceae sp., Rutaceae sp. (e.g. lemons, oranges, mandarins and grapefruit); Solanaceae sp. (e.g. tomatoes, potatoes, peppers, capsicum, aubergines, tobacco), Liliaceae sp., Compositae sp. (e.g. lettuce, artichokes and chicory—including root chicory, endive or common chicory), Umbelliferae sp. (e.g. carrots, parsley, celery and celeriac), Cucurbitaceae sp. (e.g. cucumbers—including gherkins, pumpkins, watermelons, calabashes and melons), Alliaceae sp. (e.g. leeks and onions), Cruciferae sp. (e.g. white cabbage, red cabbage, broccoli, cauliflower, Brussels sprouts, pak choi, kohlrabi, radishes, horseradish, cress and chinese cabbage), Leguminosae sp. (e.g. peanuts, peas, lentils and beans—e.g. common beans and broad beans), Chenopodiaceae sp. (e.g. Swiss chard, fodder beet, spinach, beetroot), Linaceae sp. (e.g. hemp), Cannabeacea sp. (e.g. cannabis), Malvaceae sp. (e.g. okra, cocoa), Papaveraceae (e.g. poppy), Asparagaceae (e.g. asparagus); useful plants and ornamental plants in the garden and woods including turf, lawn, grass and Stevia rebaudiana; and in each case genetically modified types of these plants.

Crop plants can be plants which can be obtained by conventional breeding and optimization methods or by biotechnological and genetic engineering methods or combinations of these methods, including the transgenic plants and including the plant varieties which can or cannot be protected by varietal property rights. Plants should be understood to mean all developmental stages, such as seeds, seedlings, young (immature) plants up to mature plants. Plant parts should be understood to mean all parts and organs of the plants above and below ground, such as shoot, leaf, flower and root, examples given being leaves, needles, stalks, stems, flowers, fruit bodies, fruits and seeds, and also tubers, roots and rhizomes. Parts of plants also include harvested plants or harvested plant parts and vegetative and generative propagation material, for example seedlings, tubers, rhizomes, cuttings and seeds.

In a preferred embodiment, the germination rate of said fungal microorganism is increased by at least 5%, preferably by at least 10%, more preferably by at least 20%, even more preferably by at least 30% or even at least 40% or 50% as compared to the germination rate of the same fungal microorganism comprised in a liquid composition suspended in water in a ratio of 1:99. Increased germination rates of at least 20%, more preferably by at least 30% or even at least 40% or 50% are preferably achieved in liquid compositions which have been stored for at least 3 months.

In connection with the present invention, an “increased germination rate” refers to a germination rate of fungal spores formulated in a liquid composition essentially free of water which is at least 5%, preferably at least 10% higher than that of dormant fungal structures or organs, such as spores not treated according to the method of the present invention but treated equally otherwise (“control spores”), preferably at least 15%, more preferably at least 20% or at least 30% and most preferably at least 50% higher. In some cases, a germination rate is even increased by at least 70% or 80% or 100%. The germination rate may be in particular increased after storage of a formulation for at least 8 months at 30° C. as compared to a formulation immediately after production. Accordingly, in one embodiment, said increased germination rate is observed after 8 months of storage at 30° C., preferably already after 6 months, more preferably already after 4 months. In such cases, the germination rate may be even further increased as compared to a sample not treated according to the invention, i.e. increased by at least 25%, at least 30%, at least 50% or at least 80%. Preferably, the control method comprises suspension of the spore containing formulation in a water-based liquid in a ratio of 1:99. In other words, “increased germination rate” means a germination rate of at least 105%, preferably at least 110% of that of control spores, preferably at least 115%, more preferably at least 120% or at least 130% and most preferably at least 150% or higher after 3 months of storage at 30° C. Preferably, said improved germination rate is still visible or even increased until at least 4 months of storage at 30° C., more preferably at least 6 months and most preferably at least 9 months of storage at 30° C., such as at least 8 months, at least 10 months or even 12 months or more. With respect to the above percentages, these are taken based on the germination rate observed at a given time for a sample not treated according to the invention.

In a preferred embodiment, the liquid composition is essentially free of or does not contain carboxylic acid triglycerides from vegetable oils. Such carboxylic acid triglycerides comprise glycerol bound to fatty acids, wherein the term “fatty acid” relates to linear carboxylic acids having 12-18 C-atoms. Such vegetable oils comprise e.g. and preferably consist of those which are liquid at room temperature, such as com oil, sunflower oil, soybean oil, rapeseed oil, peanut oil, cottonseed oil, rice bran oil, safflower oil, olive oil, linseed oil and castor oil. The skilled person is aware of which carboxylic acid triglycerides may be found in vegetable oils. A definition of vegetable oils may be found at https://en.wikipedia.org/wikiNegetable_oil (as on Jul. 20, 2018), and a summary of such carboxylic acid triglycerides may be found at http://www.dgfett.de/material/fszus.php (as on Jul. 20, 2018). Concentrations of carboxylic acid triglycerides of between 0.3 wt.-% and 8 wt.-%, such as between 0.3 wt.-% and 5 wt.-%, or between 4 wt.-% and 7 wt.-% fall within the definition of “essentially free of carboxylic acid triglycerides”. Accordingly, exemplary concentrations of carboxylic acid triglycerides include 1%, 2%, 3%, 4%, 5%, 6%, 7% and 8% which all fall within the definition of “essentially free of carboxylic acid triglycerides”. In other words, “essentially free of carboxylic acid triglycerides” means a content in the liquid formulation of 8% or less, preferably 7% or less, even more preferably 5% or less of carboxylic acid triglycerides.

In another preferred embodiment, the composition is essentially free of mineral oil. The term “essentially free of mineral oil” or “essentially free of oil” as described further below refers to a content of oil of less than 5 wt.-%, preferably less than 4 wt.-%, even more preferably less than 3 wt.-% and most preferably less than 2 wt.-% such as 1 wt.-%, 0.1 wt.-%, 0.05 wt.-% or even 0.01 wt.-%. It cannot be excluded that the liquid composition of the present invention contains traces of oil due to the production process of its ingredients. The liquid composition as used herein does not contain oil except for such traces.

In one embodiment, the composition is essentially free of oil. In connection with the present invention, oil shall be defined as any liquid which is essentially not water-miscible or self-emulsifyable in water, e.g. paraffinic oils, fatty acid triglycerides, fatty acid monoesters, certain silicone oils, aromatic solvents or other water-immiscible organic solvents, but not polyether-modified trisiloxanes. Ingredients such as polyether-modified trisiloxane would normally be seen as an oil according to the above definition of oil (e. g. a silicone oil). However, it is understood that polyether-modified trisiloxane is explicitly not seen as oil within the meaning of the present invention. In this embodiment, the liquid composition is accordingly free of at least fatty acid triglycerides and mineral oil with the exception of polyether-modified trisiloxanes.

In another embodiment, the liquid composition contains carboxylic acid triglycerides from vegetable oils as described above, and preferably no other oil as defined above. Alternatively or in addition, the liquid composition also contains mineral oil as described above.

The examples illustrate the invention in a non-limiting fashion.

### EXAMPLE 1

### Materials and Methods

### Production of Spores and Spore Formulations

Fermentation of P. lilacinum strain 251 and I. fumosorosea strain Apopka 97 was carried out in a modular solid state fermenter as described in US6620614. Harvested spores were dried to a residual moisture content of <10%. P. lilacinum strain 251 spores were dispersed in BreakThru S245 (final formulation comprising about 74 wt.-% BreakThruS240 and about 6% Aerosil) yielding formulations containing >5.5E+10 spores/ml. I. fumosorosea strain Apopka 97 spores were dispersed in Tween 20 (final formulation comprising about 95 wt.-% Tween 20, about 2.5wt.-% Aerosil 200 and about 2.5% spores) yielding formulations containing >5.0E+09 spores/ml.

### Determination of the Germination Rate

To determine the germination rate of fungal spores, water-based spore suspensions were generated. If not otherwise indicated, for the control samples, the liquid composition (formulated spores) was suspended with pure water in a ratio of composition and water of 1:99. Both the water and the composition were equilibrated to 22° C. before mixing. Also the next steps were conducted at 22° C. The suspensions were stirred with magnetic stir bars for at least 15 minutes (700 rpm) before spore suspensions were further diluted in water and spread on PDA (potato dextrose agar) plates or PDA plates containing 20 μg/ml benomyl. The plates were incubated at 25° C. for 1 to 2 days until germination was monitored microscopically. The fungistatic compound benomyl which does not inhibit spore germination but inhibits hyphal elongation allows tracking spore germination after prolonged incubation times because the not germinated spores are not overgrown by hyphae of the germinated spores. To test the influence of the ratio of liquid composition and water on the ability of the spores to germinate, the liquid composition was suspended with varying volumes of pure water (see examples below). If not otherwise indicated, both the water and the composition were equilibrated to 22° C. before mixing. Such a suspension, hereinafter also called “imbibition premix” was then further diluted with 22° C. temperated water to reach the same ratio of composition and water as in the control samples, i.e 1:99. The subsequent processing of samples derived from an imbibition premix were the same as those for the controls.

### EXAMPLE 2

### The Ratio of Liquid Composition and Water During Imbibition Impacts the Germination Rate of Formulated Spores

Spores of P. lilacinum strain 251 were produced, dried, and formulated in BreakThru S245 as described in Example 1. Eventually, batches of formulated spores were stored for prolonged time at 30° C. as indicated in Table 1. From the formulated spore batches several imbibition premixes were created ranging from a ratio of liquid composition and water from 1000:1 to 1:10. The premixes were held for two hours at 22° C. before they were diluted to the same ratio of liquid composition and water as in the control (see Example 1). The ability of the spores to germinate was then monitored after 1 day incubation on PDA as described in Example 1. Compared to the control, in which the spore suspension was created with a ratio of liquid composition and water of 1:99, imbibition premixes of bigger ratios could improve the germination rate by more than 90% (Table 1). Ratios of liquid composition and water between 100:1 and 1:5 yielded particularly improved germination rates.

TABLE 1
The ratio of liquid composition and water during imbibition impacts the germination rate of formulated spores.

| | formulated spores | | formulated spores | | formulated spores | | formulated spores | |
|---|---|---|---|---|---|---|---|---|
| | batch #1 | | batch #2 | | batch #3 | | batch #4 | |
| | 8 months @ 30° C. | | 8 months @ 30° C. | | 10 months @ 30° C. | | 0 months storage | |
| ratio liquid | Germination^{a} | STD | Germination^{a} | STD | Germination^{a} | STD | Germination^{a} | STD |
| composition:water | (%) | Ev^{b} | (%) | EV^{b} | (%) | EV^{b} | (%) | EV^{b} |
| 1000:1 | NA | NA | NA | NA | 64.4 | 0.8 | NA | NA |
| 333:1 | NA | NA | NA | NA | 67.4 | 0.1 | NA | NA |
| 100:1 | 63.8 | 7.6 | 54.9 | 1.6 | 74.0 | 1.8 | 88.6 | 0.1 |
| 33:1 | 85.6 | 1.1 | 82.0 | 1.9 | 79.5 | 1.3 | 94.4 | 1.0 |
| 10:1 | 91.5 | 0.0 | 87.5 | 2.5 | 80.2 | 0.2 | 97.6 | 0.7 |
| 1:1 | 79.3 | 0.9 | 79.9 | 1.1 | 77.0 | 0.1 | 92.5 | 0.1 |
| 1:5 | 78.8 | 0.5 | 68.8 | 1.1 | NA | NA | 83.8 | 1.0 |
| 1:10 | 69.6 | 1.7 | 48.3 | 0.3 | NA | NA | 66.7 | 3.2 |
| control | 47.8 | 2.9 | 57.4 | 3.7 | 51.1 | 2.6 | 82.4 | 0.8 |

^{a}Average of two replicates;
^{b}Standard deviation of two replicates;
the notation “NA” denotes that the data point was not determined

### EXAMPLE 3

### Impact of Imbibition Duration on the Germination Rate of Formulated Spores

Spores of P. lilacinum strain 251 were produced, dried, and formulated in BreakThru S245 as described in Example 1. Eventually, batches of formulated spores were stored for prolonged time at 30° C. as indicated in Table 2. Imbibition premixes were prepared as described in Example 1, and the premixes were held for various durations at 22° C. as shown in Table 2. The ability of the spores to germinate was then monitored after 1 day incubation on PDA as described in Example 1. The results of this example demonstrate that short imbibitions of 1 minute in an imbibition premix are sufficient to increase the germination rate by more than 80% compared to control imbibitions (Table 2).

TABLE 2
Impact of imbibition duration at various liquid composition:
water ratios on the germination rate of formulated spores.

| | imbibition | formulated spores #3 | |
|---|---|---|---|
| ratio liquid | premix | 10 months @ 30° C. | |
| composition: | holding time | Germination^{a} | |
| water | (min) | (%) | STDEV^{b} |
| 10:1 | 1 | 52.9 | 0.5 |
| | 5 | 65.8 | 1.6 |
| | 30 | 63.3 | 1.0 |
| 1:1 | 1 | 65.9 | 1.8 |
| | 5 | 64.6 | 1.1 |
| | 30 | 48.6 | 0.6 |

| | | |
|---|---|---|
| Control | 36.0 | 0.8 |

^{a}Average of two replicates;
^{b}Standard deviation of two replicates

### EXAMPLE 4

### Impact of Imbibition Temperature on the Germination Rate of Formulated Spores

Spores of P. lilacinum strain 251 were produced, dried, and formulated in BreakThru S245 as described in Example 1. Eventually, batches of formulated spores were stored for prolonged time at 30° C. as indicated in Table 3. Imbibition premixes were prepared essentially as described in Example 1, but in this example, sub samples of a batch of formulated spores were equilibrated to the temperatures 4° C., 12° C., 22° C. and 37°. The water used for the respective imbibition was equilibrated to the same temperatures, and only equally temperated liquid compositions and water were mixed. Thus, the initial temperature for preparing the imbibition premix corresponded to 4° C., 12° C., 22° C. and 37° C., as indicated in Table 3. However since the mixing and the subsequent handling of the samples occurred at an surrounding temperature of 22° C. the temperature of the samples most likely changed towards 22° C. over time. This assumed temperature shift was not monitored. All premixes were hold for 30 min before the ability of the spores to germinate was monitored after 1 day incubation on PDA as described in Example 1. The results showed that warmer imbibitions increased the germination rate compared to colder imbibitions (Table 3). Compared to control imbibitions, which were conducted at 22° C., an imbibition premix at 12° C. already yielded an increased germination rate by 36%; the germination rate could be even further increased by preparing the imbibition premix at temperatures of 22° C. and above (Table 3).

TABLE 3
Impact of imbibition temperature of various liquid composition:
water ratios on the germination rate of formulated spores.

| | | formulated spores | |
|---|---|---|---|
| | initial temperature | 10 months @ 30° C. | |
| ratio liquid | of imbibition | Germination^{a} | |
| composition:water | premix | (%) | STDEV^{b} |
| 10:1 | 4° C. | 53.4 | 0.7 |
| | 12° C. | 69.9 | 1.7 |
| | 22° C. | 72.6 | 4.7 |
| | 37° C. | 77.2 | 2.6 |
| 1:1 | 4° C. | 56.3 | 0.4 |
| | 12° C. | 70.5 | 1.8 |
| | 22° C. | 76.1 | 2.8 |
| | 37° C. | 77.8 | 1.1 |

| | | |
|---|---|---|
| control | 51.1 | 2.6 |

^{a}Average of two replicates;
^{b}Standard deviation of two replicates

### EXAMPLE 5

### Spore Content in Liquid Composition Hardly Influences the Germination Rate

Spores of P. lilacinum strain 251 were produced, dried, and formulated in BreakThru S245 as described in Example 1. Eventually, batches of formulated spores were stored for prolonged time at 30° C. as indicated in Table 4. Sub samples of a batch of formulated spores were further diluted with BreakThru S245 to lower the fraction of spores in the formulation from 20% to 10% and 2%, after which imbibition premixes were prepared as shown in Table 4 and described in Example 1. As imbibition control served the original composition with 20% spore fraction. All imbibitions were conducted at 22° C. and the premixes were held for 30 min before the ability of the spores to germinate was monitored after 1 day incubation on PDA as described in Example 1. The results did not reveal clear hints that the amount of spores in the formulation determines the ability of the imbibition premix to increase the germination rate: compared to the control, the premixes with a ratio of liquid composition and water of 1:1 increased the germination by 79%-100% and the premixes with a ratio of liquid composition and water of 10:1 increased the germination by 109%-123% (Table 4). From this data, it can be concluded that rather the ratio of liquid composition and water is decisive for the increased germination and not the ratio of spores and water.

TABLE 4
Impact of the spore fraction in
formulation on the ability to germinate.

| fraction of | | formulated spores | |
|---|---|---|---|
| spores in | ratio liquid | 9 months @ 30° C. | |
| formulation | composition: | Germination^{a} | |
| (%) | water | (%) | STDEV^{b} |
| 20% | 10:1 | 76.9 | 1.1 |
| 10% | | 81.8 | 3.1 |
| 2% | | 76.8 | 0.0 |
| 20% | 1:1 | 65.8 | 0.4 |
| 10% | | 73.4 | 0.8 |
| 2% | | 68.3 | 3.1 |

| | | |
|---|---|---|
| control | 36.7 | 3.2 |

^{a}Average of two replicates;
^{b}Standard deviation of two replicates

### EXAMPLE 6

### The Ratio of Liquid Composition and Water During Imbibition Impacts the Germination Rate of I. Fumosorosea Spores Formulated in the Surfactant Tween 20

Spores of I. fumosorosea strain Apopka 97 were produced, dried, and formulated in the carrier liquid Tween 20 as described in Example 1. The formulated spores were stored in closed bottles at 40° C. for 2 months before subjecting them to quality control. Imbibition premixes were prepared as described in Example 1, and the premixes were held for 30 minutes at 22° C. before further dilution. The ability of the spores to germinate was then monitored after 1 day incubation on PDA. The results of this example demonstrate that imbibition premixes of ratios of liquid composition and water of 10:1 and 1:1 lead to improved germination rates compared to spore suspensions prepared in the control ratio of 1:99 (Table 5).

TABLE 5
The ratio of liquid composition and water during
imbibition impacts the germination rate of I. fumosorosea
spores formulated in Tween 20 after storage.

| | formulated spores | |
|---|---|---|
| | 6 months storage | |
| ratio liquid | at 30° C. | |
| composition: | Germination^{a} | |
| water | (%) | STDEV^{b} |
| 10:1 | 89.3 | 2.0 |
| 1:1 | 91.2 | 0.4 |
| control | 86.1 | 0.1 |

^{a}Average of two replicates;
^{b}Standard deviation of two replicates;
the notation “NA” denotes that the data point was not determined

### EXAMPLE 7

### The Ratio of Liquid Composition and Water During Imbibition Impacts the Germination Rate of M. Brunneum Spores Formulated in Petroleum Distillates

A commercial product containing >2E+09 spores/g of M. brunneum strain F52 formulated in a petroleum based liquid was stored for prolonged time of 12 months at 20° C. after which the bottle has been opened and closed under normal atmosphere, followed by storage of another 14 months at 4° C. Thus, total storage time was 26 months before subjecting them to quality control. Imbibition premixes were prepared as described in Example 1, and the premixes were held for 30 minutes at 22° C. before further dilution. The ability of the spores to germinate was then monitored after 1 day incubation on PDA. Compared to control imbibitions at a ratio of liquid composition and water of 1:99, imbibition premixes of ratios of 1:1 and in particular 10:1 improved germination rates (Table 6).

TABLE 6
The ratio of liquid composition and water during imbibition
impacts the germination rate of M. brunneum spores
formulated in petroleum distillates after prolonged storage.

| | formulated spores | |
|---|---|---|
| ratio liquid | 26 months storage | |
| composition: | Germination^{a} | |
| water | (%) | STDEV^{b} |
| 10:1 | 37.2 | 3.0 |
| 1:1 | 27.3 | 2.6 |
| control | 9.8 | 0.2 |

^{a}Average of two replicates;
^{b}Standard deviation of two replicates;
the notation “NA” denotes that the data point was not determined

1. A method of increasing the germination rate of spores of a fungal microorganisms formulated in a liquid composition essentially free of water, comprising, prior to applying to a plant, plant part or locus where a plant is growing or intended to be grown, suspending said composition in an agriculturally acceptable water-based liquid in a ratio of liquid composition:water-based liquid of between 1000:1 and 1:10.

2. A method of increasing efficacy of a biological control agent based on spores of a fungal microorganisms and formulated in a liquid composition essentially free of water, comprising, prior to applying to a plant, plant part or locus where a plant is growing or intended to be grown, suspending said composition in an agriculturally acceptable water-based liquid in a ratio of liquid composition:water-based liquid of between 1000:1 and 1:10.

3. A method of controlling the quality of a biological control agent based on spores of a fungal microorganism formulated in a liquid composition essentially free of water, comprising suspending a sample of said composition in a water-based liquid in a ratio of liquid composition:water-based liquid of between 1000:1 and 1:10 and cultivating a part of the resulting suspension.

4. The method according to claim 1, wherein said spores of a fungal microorganism are conidia.

5. The method according to claim 1, wherein said liquid composition is essentially free of carboxylic acid triglycerides.

6. The method according to claim 1, wherein said liquid composition comprises at least one surfactant.

7. The method according to claim 6, wherein said liquid composition comprises at least 1%, preferably at least 5 wt.-% of said at least one surfactant.

8. The method according to claim 7, wherein said liquid composition comprises at least 50 wt.-% of said at least one surfactant.

9. The method according to claim 3, wherein said water further comprises an agent which decreases or prevents hyphal growth.

10. The method according to claim 9, wherein said agent is benomyl.

11. The method according to claim 1, wherein said suspension is to be kept for at least 10 seconds, preferably at least 1 minute prior to application or cultivation.

12. The method according to claim 3, wherein the ratio of composition and water is between 50:1 and 1:5.

13. The method according to claim 12, further comprising, after said suspending, further diluting the suspension.

14. The method according claim 6, wherein said surfactant is a polyether-modified trisiloxane.

15. The method according to claim 14, wherein said surfactant is BREAKTHRU® S240 or BREAKTHRU® S245.

16. The method according claim 1, wherein said fungal microorganism is useful in agriculture.

17. The method according to claim 1, wherein the germination rate of said fungal microorganism is increased by at least 5% as compared to the germination rate of the same fungal microorganism comprised in a liquid composition suspended in a water-based liquid in a ratio of 1:99.

18. The method according to claim 1, wherein said fungal microorganism is Purpureocillium lilacinum.

19. The method according to claim 1, wherein said liquid composition and/or the water based liquid have a temperature of at least 12° C.

20. The method according to claim 1, wherein said liquid composition is essentially free of mineral oil.

21. The method according to claim 1, wherein said liquid composition is essentially free of oil.
